(19) United States
(12) Plant Patent Application Publication
(10) Pub. No.: US 2009/0038044 P1
(43) Pub. Date: Feb. 5, 2009

(54) Syzygium floribundum plant named 'DOW20'

(76) Inventor: Todd Anthony Layt, Richmond (AU)

Correspondence Address: Sam R. McCoy Jr., P.O. Box 2108, Mount Pleasant, SC, 29465, US

(21) Appl. No.: 11/891,491

(22) Filed: Aug. 10, 2007

Related U.S. Application Data
(60) Provisional application No. 60/836,878, filed on Aug. 10, 2006.

(51) Int. Cl. A01H 5/00

(52) U.S. Cl. PLT/263.1

(57) ABSTRACT

‘DOW20’ is a distinctive variety of Syzygium floribundum, which is characterized by its combination of light green immature leaf color, green new stem color, strong undulation of the leaf margin and strongly weeping plant growth habit.

### LATIN NAME OF THE GENUS AND SPECIES

The Latin name of the novel variety disclosed herein is Syzygium floribundum ‘DOW20’.

Prior to a taxonomic review in 2006 the Latin name was Waterhousea floribunda (Reference: Craven, L.A., Biffin, E., Ashton, P.S. (2006). Acmena, Acmenospora, Cleistocalyx, Piliocalyx and Waterhousea formally transferred to Syzygium (Myrtaceae). Blumea 51: 131-142.

### VARIETY DENOMINATION

The inventive variety of Syzygium floribundum disclosed herein has been given the variety denomination ‘DOW20’.

### BACKGROUND OF THE INVENTION

The present invention relates to a new and distinct variety of evergreen perennial Syzygium floribundum, which has been named ‘DOW20’. Syzygium floribundum is a small to large Australian native tree that is used as an ornamental tree in landscape and amenity horticulture. Syzygium floribundum has a weeping plant growth habit. Large glossy green leaves are arranged opposite and the bark on the stems is a dark grey to brown color. Flowers are white and arranged in panicles, appearing in spring and are followed by firm berries that are green in color.

An application for plant breeders' rights for ‘DOW20’ has been lodged with the Australian Plant Breeders Rights Office, and was accepted on 29 April 2006 (under Application No. 2005/289).

Parentage: The cultivar ‘DOW20’ was discovered in 2001 in Tuckombil, New South Wales, Australia, during a seedling selection of cultivated Syzygium floribundum (unpatented). The parent form is characterized by a medium weeping plant growth habit, brown new stem color, a reddish immature leaf color and weak undulation of the leaf margin. Selection criteria for ‘DOW20’ were light green immature leaf color, green new stem color, strong undulation of the leaf margin and strongly weeping plant growth habit.

Asexual reproduction. The new variety ‘DOW20’ was first asexually propagated by vegetative cuttings in the state of New South Wales, Australia in 2001. The distinctive characteristics of cultivar ‘DOW20’ have remained stable and true to type through successive cycles of asexual propagation.

### SUMMARY OF THE INVENTION ‘DOW20’ is a distinctive variety of Syzygium floribundum, which is characterized by its light green immature leaf color, green new stem color, strong undulation of the leaf margin and strongly weeping plant growth habit.

### BRIEF DESCRIPTION OF THE DRAWINGS

FIG. 1 shows a Syzygium floribundum ‘DOW20’ plant at approximately 30-months of age. The plant was propagated in a greenhouse and was transferred to the field at six months and grown out in full sun.

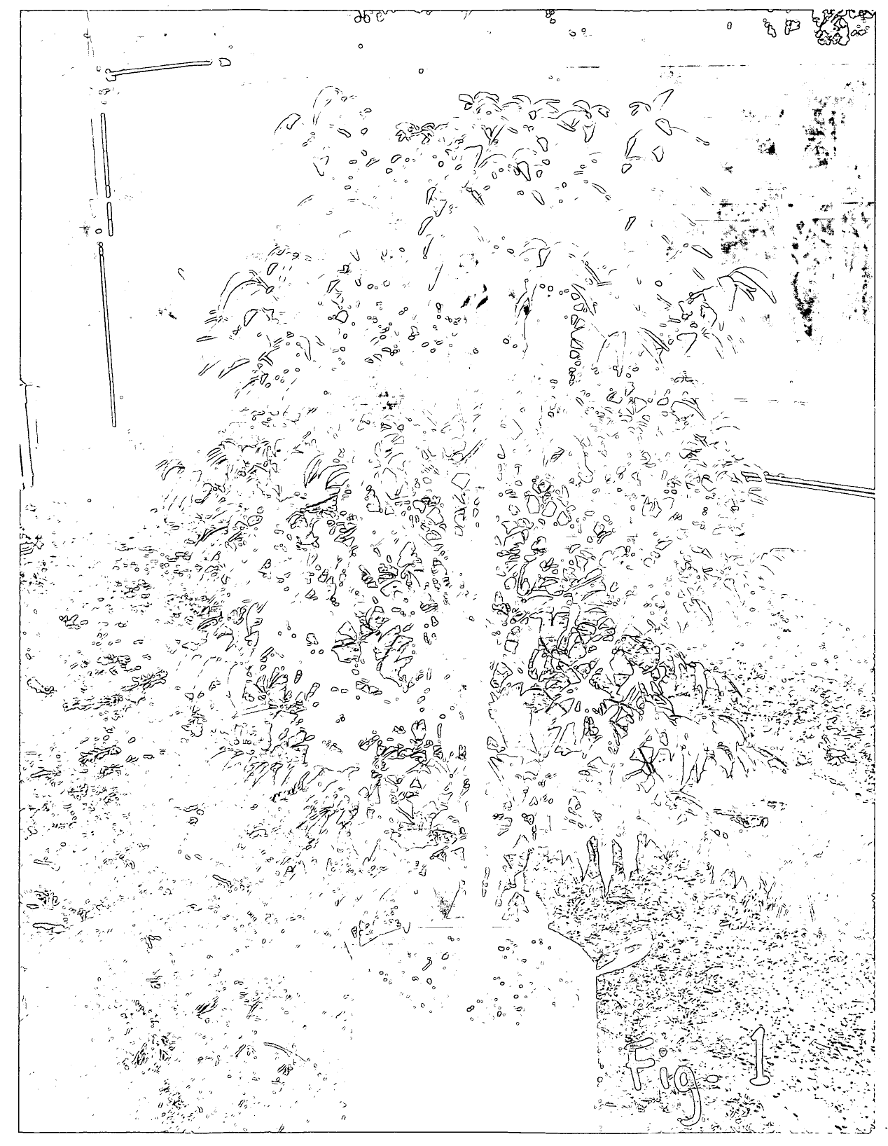

FIG. 2 shows the colors and habit of the foliage of ‘DOW20’.

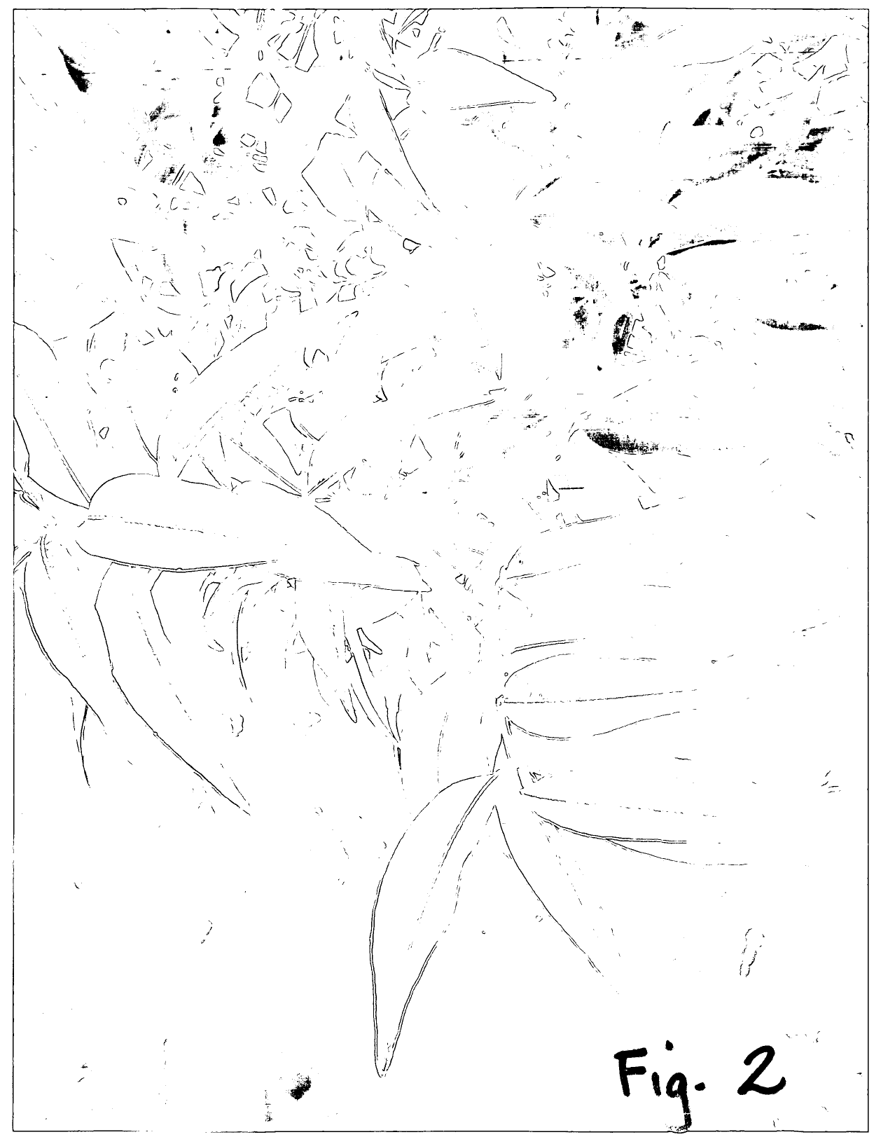

FIG. 3 shows the upper and lower side colors and shapes of leaves of ‘DOW20’.

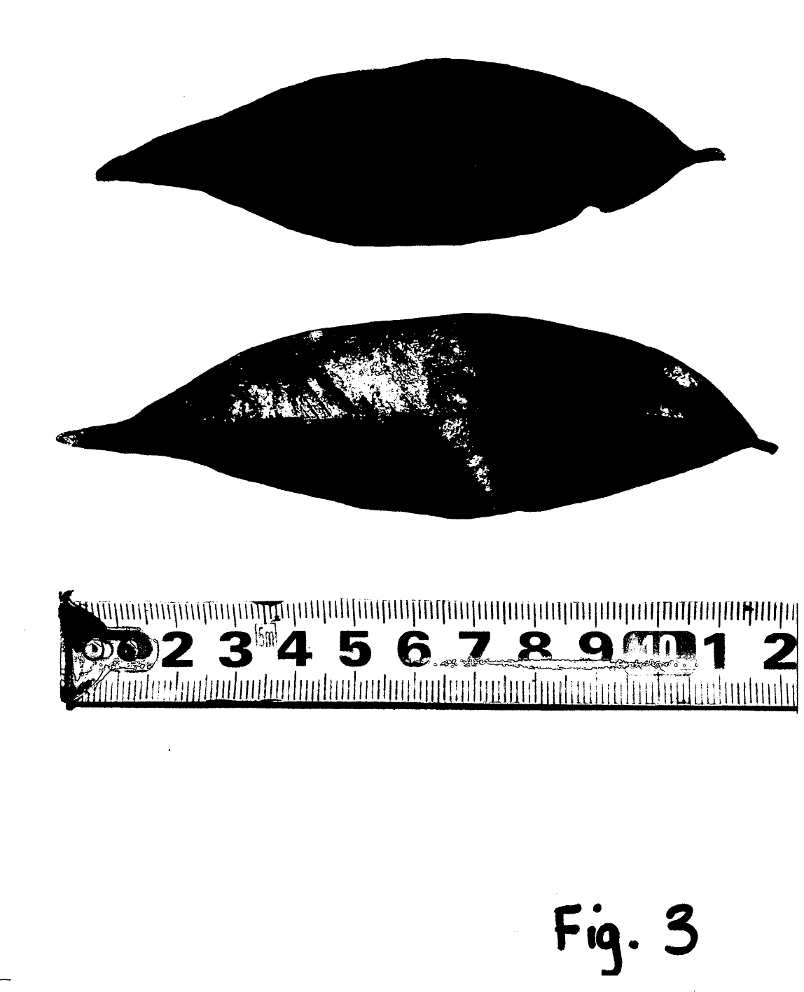

### DETAILED BOTANICAL DESCRIPTION OF THE VARIETY

The following is a detailed botanical description of a new and distinct variety of Syzygium floribundum known as ‘DOW20’ based upon observations of 30-month old plants grown in nursery pots in full sun in open beds in Theresa Park, New South Wales, Australia during 2005- 2006 (as described below in connection with Table 1). Plant observations and descriptions were taken in winter 2006.

Those skilled in the art will appreciate that certain characteristics will vary with older or, conversely, with younger plants. ‘DOW20’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 1995 edition. ‘DOW20’ is an evergreen perennial Syzygium floribundum. ‘DOW20’ is an spreading tree with a strongly weeping growth habit and a vigorous growth rate. ‘DOW20’ has a light green immature leaf color light (expanding new growth). ‘DOW20’ has a green new stem color. ‘DOW20’ has an elliptic leaf shape with an acuminate leaf apex shape and strong leaf margin undulation. The surface of the leaf upper side of ‘DOW20’ has a glossy, dark green appearance and an absence of pubescence. The surface of the leaf lower side of ‘DOW20’ has a dull, lighter green appearance and an absence of pubescence. These characteristics are unusual for Syzygium floribundum, as these plants usually have a medium weeping plant growth habit, brown new stem color, a reddish immature leaf color (new growth flush) and weak undulation of the leaf margin. A botanical description of ‘DOW20’ and comparisons with the parent form of Syzygium floribundum are provided below.

### Technical Description of the Variety

Plant characteristics: Growth habit spreading and strongly weeping, height medium (not yet recorded for a fully mature tree), typical of the species.
Stems: New stem has a smooth surface texture, color of young stem yellow green (RHS 153B), gradually changing with age to yellow green (RHS 152A-B). The old bark on old stems and the trunk has an overall grayed brown appearance composed of a mixture of brown (RHS 200A), particularly around bud and shoot scar marks, grayed brown (RHS 199D) and grayed green (ca RHS 197C-D).
Leaves: Attitude horizontal to weeping, length medium (range 11 to 12 cm), width broad (range 3.5 to 4.2 cm), leaf upper side color yellow green (RHS 147A) with strong surface glossiness, mature leaf lower side color yellow green (RHS 146C), variegation absent, shape elliptic, apex acuminate, cross section flat, margin entire, prominent margin undulation, upper side midrib color yellow green (RHS 154C-D), lower side midrib color yellow green (RHS 152D), venation pinnate and corresponding to leaf color, petiole length short-medium (range 8-10 mm).
Flowers and fruiting: Flowering characteristics of ‘DOW20’ have yet to be recorded.
Cold and heat tolerance: ‘DOW20’ has not yet been observed under all conditions. ‘DOW20’ is heat tolerant. It adapted well to the high heat conditions greater than 40 degrees Celsius during summer in Theresa Park, New South Wales, Australia without any noticeable damage. It has survived frosts to minus 4 degrees Celcius. Testing for environmental tolerances is ongoing.
Drought tolerance: Good drought tolerance typical of the species once established. Prefers moist condition, can survive wet conditions with adequate drainage, but not boggy conditions. ‘DOW20’ has not yet been observed under all conditions. Testing for environmental tolerances is ongoing.
Pest resistance: No known pests.
Cultural conditions: ‘DOW20’ can tolerate low nutrient conditions; it does not like continually wet soil conditions, but can tolerate well-draining sandy soils to very heavy clay soils.

### Comparisons with Other Syzygium floribundum.

‘DOW20’ is a more attractive ornamental tree as compared with the parent Syzygium floribundum (unpatented, wild type). ‘DOW20’ is characterized by its light green immature leaf color, green new stem color, strong undulation of the leaf margin and strongly weeping plant growth habit as compared with Syzygium floribundum. The appearance of ‘DOW20’ is lush with weeping foliage with dark green crinkled leaves, whereas Syzygium floribundum has more red foliage in the new growth flush, less crinkled leaves, browner stems and a lesser weeping habit (see Table 1).

The Syzygium floribundum (unpatented, wild type) is the most similar comparator variety to ‘DOW20’.

A comparative trial of Syzygium cultivar‘DOW20’ with Syzygium floribundum (unpatented, wild type) is currently being carried out in Theresa Park, New South Wales, Australia. The initial data are presented in Table 1 below.

TABLE 1

| | Syzygium Variety | |
|---|---|---|
| Characteristic | ‘DOW20’ | Syzygium floribundum |
| Plant: growth habit | strongly weeping | medium weeping |
| Stem: new color | green | brown |
| Leaf: immature color | green | red |
| Leaf: margin undulation | strong | medium |

That which is claimed is:

1. A new and distinct variety of Syzygium floribundum plant named ‘DOW20’, substantially as described and illustrated herein.
